(19) United States
(12) Patent Application Publication
(10) Pub. No.: US 2009/0048553 A1
(43) Pub. Date: Feb. 19, 2009

(54) Bone marrow transplantation for preventing and treating neurological conditions and diabetes

(76) Inventors: Lixian Jiang, Tampa, FL (US); Calvin Cao, Tampa, FL (US)

Correspondence Address: Lixian Jiang;Suite 901, 2203 North Lois Ave, Tampa, FL, 33607, US

(21) Appl. No.: 11/893,305

(22) Filed: Aug. 15, 2007

(51) Int. Cl. A61M 37/00

(52) U.S. Cl. 604/6.12

(57) ABSTRACT

The present discloses a method to treat stroke, spinal cord injury, Parkinson disease, multiple sclerosis (MS), Parkinson disease, Amyotrophic Lateral Sclerosis (ALS), and other neurological as well as diabetes with bone marrow transplantation. The said bone marrow is transplanted as a whole, and does not need any cell isolation and cell expansion.

### FIELD OF THE INVENTION

The present invention relates generally to the field of stroke, spinal cord injury, multiple sclerosis (MS), Parkinson disease, Amyotrophic Lateral Sclerosis (ALS), and other neurological conditions as well as diabetes utilizing partially or completely bone marrow cells with particular emphasis on cell replacement with stem cells in bone marrow tissues. Different from any other bone marrow transplantation technology which needs cell isolation and cell expansion before transplantation, the present invention discloses a new technology which circumvents these procedures. Instead, the present invention just mixes with cell expansion solution and directly transfuses into patient system, which greatly reduces the medical cost, and improve the medical operation safety.

### BACKGROUND OF THE INVENTION

Bone marrow is the soft tissue found in the hollow interior of bones, and it is in rich of hematopoietic and non-hematopoietic stem cells. In adult, hematopoietic stem cells can proliferate and differentiate into all type of blood cells, and provide blood cells to system. On the other hand, non-hematopoietic stem cells can proliferate and differentiate into all type of cells in system, for example muscles cells. In addition, cells from bone marrow could differentiate into neural-like cells, which could replace the cell loss in stroke, spinal cord injury and other neurological conditions. Experimental study revealed that stroked animals had neurological behavioral function improvement after bone marrow transplantation. It is estimated that every year there are around 18,000-20,000 bone marrow transplantation globally.

However, bone marrow transplantation after chemotherapy usually cause sever immune rejection. For patients with leukemia and other blood disorder, bone marrow transplantation requires HLA match. HLA stands for human leukocyte antigen system, the name of the human major histocompatibility complex (MHC). The chance of two unrelated individuals having identical HLA molecules is very low, which results in the shortage of donor tissue. This greatly impedes its wide spreads.

For stroke, spinal cord injury, multiple sclerosis (MS), Parkinson disease, Amyotrophic Lateral Sclerosis (ALS), diabetes, and other organ dysfunction, it does not need to clear up the whole system with chemotherapy before cell transplantation. Therefore, it is possible to use the patients' own bone marrow for cell transplantation, and since the donor tissue comes from patient himself, there is no matching problem. Currently, experiments demonstrate that autologous transplantation, in which the donor and recipient are the same person, can successfully improve the function recovery of animal in the model of stroke, spinal cord injury, diabetes, and other conditions. This method is repeated in clinical trial for stroke or spinal cord injury in United States, Europe, Japan and China, and the results turn out successful.

The actual part of bone marrow for cell transplantation is bone marrow stromal cells or mesenchymal cells, which can be obtained from tibia or sacral bone by aspiration. Stromal cells contain several adult stem cells. The average volume of concentrated bone marrow was 132±47 ml, and it is estimated there is 1 stem cell in 3.1×10⁴ bone marrow mononuclear cells (BMC Cell Biol. 2006; 7: 14). Apparently, the level of stem cell concentration is very low for bone marrow. However, even that bone marrow is the richest source of stem cell compared to umbilical cord blood, peripheral blood and other resource.

On the other side, to achieve a successful bone marrow transplantation, a minimal 1×10⁶ stem cells are needed for each kilogram body weight (eMedicine, Oncology, Aug. 18, 2006). Therefore, the small amount of stem cell average the stem cell definitely is not enough for bone marrow transplantation. Scientists try to expand stem cells in vitro to achieve enough cells before transplantation. The technology of bone marrow cell isolation and expansion has been developed. For example, U.S. Pat. No. 7,015,037 discloses a method to isolate stem cell from bone marrow; another U.S. Pat. No. 6,649,189 discloses a method to expand the cells in incubation.

Cell isolation and expansion can provide a large quantity and qualified donor cells for transplantation. However, this process also increases the medical expense, and makes the donor cells exposed to environment, which devastate cell safety.

The present technology discloses a method to treat stroke, spinal cord injury, Parkinson disease, multiple sclerosis (MS), Amyotrophic Lateral Sclerosis (ALS), and other neurological condition as well as diabetes with bone marrow transplantation via intravenous infusion, where bone marrow is aspirated from patients, and return back to patient directly with blood transfusion system. Growth factor and other medications were used to enhance cell migration to the target place. Once migrate into the right place, the exogenous growth factors also enhance stem cells differentiate into target cells, and replace the dysfunctional cells in patient.

### SUMMARY OF THE INVENTION

The present invention discloses a method to treat stroke, spinal cord injury, multiple sclerosis (MS), Parkinson disease, Amyotrophic Lateral Sclerosis (ALS), and other neurological condition as well as diabetes with autologous or allogeneic bone marrow transplantation via intravenous infusion. Unlike conventional bone marrow transplantation, which at least needs to isolate mononuclear cells from bone marrow, or process cell expansion, the said bone marrow transplantation use the whole piece of bone marrow immediate after extract from patient, and it does not need cell isolation and cell expansion.

In one embodiment, the bone marrow is obtained from tibia or sacral bone or other bone from patient himself or from donors at clinic doctor's discretion, and preferably it is from the same patient. In case it is unavailable from the same patient, it can be obtained from other human subject as a donor, and just minimal immune match test, blood match, is needed. In one embodiment, immediately after obtained bone marrow, the cells are transfused into patient's blood system through i.v. administration as a whole piece. The bone marrow extract should be in amount of 1 ml-400 ml, preferably 130-180 ml. The bone marrow solution contains saline or other medical solution for one skilled in the art to facilitate transfusion.

In one embodiment, the patient is pretreated with granulocyte colony-stimulating factor (G-CSF), a commercial name “NEUPOGEN”, for 1-4 days at the dose where Absolute Neutrophil Count (ANC) is less than_{—}10,000/mm³. For neurological patients, mannitol, a blood-brain-barrier (BBB) permeable medicine, is mixed with for bone marrow transfusion at a dose of 50 to 200 g for an adult in a 24-hour period. In another embodiment, growth factors are administered for favoring stem cell to differentiate into neural-like cells. The growth factors include, but not limited to neural growth factor (NGF) for one skilled in the art. As to diabetes patient, mannitol is not necessary. And the growth factors, insulin-like growth factor-1 (IGF-1), and/or hepatocyte growth factor (HGF), are administered. For one skilled in the art these growth factors can be replaced by other growth factors favoring insulin secretion cell differentiation.

The transfused stem cells will expand itself in the patient system, since physiological condition of human being provide enough nutrient support and proper micro-environment for stem cell proliferation. In one embodiment, the transfused cells will not reside in other tissue or organ since it is well known that NEUPOGEN has the effect to promote stem cell release from bone marrow. It has been determined in our experiment that NEUPOGEN will prevent transfused stem cell adherent to soft tissue, and keep them circulation in blood system. In another embodiment, neural growth factor and other similar growth factor promote stem cell differentiate into neural like cells, under the “homing” effect, the transfused stem cells will migrate into the injury site of central nerve system (CNS). The transient opening of BBB under the effect of mannitol will facilitate cell across the border between blood system and CNS. Once get to the injury site, transfused stem cell can differentiate into neural like cells, and replace the cell loss in the injury.

In the case of diabetes, neural growth factor is substituted by other growth factor for one skilled in the art. For example, insulin-like growth factor-1 (IGF-1), and/or hepatocyte growth factor (HGF) are good candidates. The transfused stem cell is induced to differentiate into insulin secretion cells. Under homing effect, these differentiation direction determined cells will migrate into the injury site. Once reside at the injury site, the transfused cells will proliferate and differentiate into damaged cells. Since the transfused cells are not required to migrate into CNS, mannitol is not required.

### DETAILED DESCRIPTION OF THE INVENTION

Stroke is the third leading cause of death in the United States, and annually there are around 700,000 new or recurrent attacks occurring. As for spinal cord injury, it is estimated that the annual incidence is approximately 11,000 new cases each year, while multiple sclerosis is about one person in every 1000 citizens in United States. As for diabetes, there are around 5-10% adult American suffering from this disease. Another disease plaguing in America is Amyotrophic Lateral Sclerosis (ALS). There are millions of patients in USA suffering from ALS. However, for all these diseases, there are no effective treatments available.

Bone marrow transplantation may provide an alternative treatment for these diseases. Previous study demonstrated that bone marrow is in rich of hematopoietic and non-hematopoietic stem cells. Hematopoietic stem cells can differentiate into all type blood cells, and provide blood linage for circulation system. On the other hand, non-hematopoietic stem cells, also called mesenchymal stem cells, are most primitive cells, and have the capability to differentiate into all type of cells in the body. When cultured with some neural growth factor, these non-hematopoietic stem cells can differentiate into some neural like cells, which could replace the cell loss in brain damage. Experimental and some clinical trial studies already demonstrated that bone marrow transplantation can promote neurological and behavioral function improvement in stroke, Parkinson disease, spinal cord injury, Amyotrophic Lateral Sclerosis (ALS), and multiple sclerosis as well as diabetes.

However, mesenchymal stem cells are pretty low in bone marrow, and only take 0.01% to 0.001% of the mononuclear fraction of bone marrow. On the other hand, at least one million stem cells per 1 kilogram body weight are required for bone marrow transplantation. Apparently, it is unlikely for this low quantity of the stem cells to promote functional recovery in bone marrow transplantation. A prevailing idea to solve this dilemma is to isolate mononuclear portion containing mesenchymal stem cells from bone marrow, and expand cells prior transplantation. However, this no doubt increases the medical cost and medical contamination risk.

A typical cell expansion in cell culture needs serum and other growth factors. For cell preparation to be used in clinic, serum culture is risky since it can transfer hepatitis B, HIV, and other diseases. For example, scientists found many diseases which did not know before are transferred by serum, and it is highly possible to transfer other unidentified diseases through serum. In the past years, scientist developed serum free medium for stem cell culture in preparation of clinical solution (Invitrogen Inc.). However, this greatly increases medical cost, and also it has been demonstrated that stem cells do not grow well in serum free medium as they do in serum culture. Therefore, serum is necessary for stem cell growth and proliferation.

Besides that, it is well known that appropriate temperature and appropriate oxygen concentration is a must for cell culture. All these cell culture parameters could be achieved in incubator. From different point of view, human blood circulation system has enough growth factor and nutrition to support cell growth and proliferation. In addition, blood circulation system has appropriate temperature and appropriate micro-environment for cell growth. Therefore, the blood system can be treated as an incubator with an automatic renewing system. We proposed that under appropriate condition which is disclosed in the present invention, the transfused bone marrow mesenchymal cells can proliferate into large amount of stem cell in blood circulation system, and migrate into the injury site. Once get the injury site, the transfused cells can replace the cell loss caused by injury, and play function over there. Therefore, it is not necessary to isolate mononuclear portion from bone marrow, and expand the cells. This invention have been incorporated into the method of treatment of stroke, spinal cord injury, multiple sclerosis (MS), Parkinson diseases, Amyotrophic Lateral Sclerosis (ALS), and other neurological condition as well as diabetes. Our experimental and clinic trial data demonstrated this method worked better than conventional bone marrow transplantation.

In one embodiment, the patients receives NEUPOGEN at the dose where Absolute Neutrophil Count (ANC) is less than_{—}10,000/mm³ for 1-4 days, preferable ANC is close to 10,000/mm³. Following that, bone marrow is obtained from tibia or sacral bone or other bone from patient himself or from donors by aspiration at clinic doctor's discretion, and preferably it is from the same patient. The bone marrow extract should be in amount of 1 ml-400 ml, preferably 130-180 ml. In another embodiment, the bone marrow extract is mixed with 1000 ml saline solution. The mixture solution drops into patient's blood system through i.v. administration within 2 hours. According to patient's condition, the mixture solution contains other medication for bone marrow transfusion.

In one embodiment, for stroke, spinal cord injury, multiple sclerosis (MS), Parkinson disease, Amyotrophic Lateral Sclerosis (ALS), and other neurological condition, mannitol, a blood-brain-barrier (BBB) permeable medicine, is mixed with for bone marrow transfusion at a dose of 50 to 200 g for an adult in a 24-hour period, preferably the patient is pretreated with mannitol for 1-3 days. In another embodiment, the bone marrow solution is mixed with neural growth factor (NGF) or fibroblast growth factor (FGF) to promote mesenchymal stem cell differentiation into neural-like cells. The daily dosage thereof is in the range of 0.01 μg-10 mg per 1 kg of the body weight in an adult, preferably in the range of 0.1 μg-1 mg/kg. The daily dosage of FGF is around 60-200 mcg/kg/day, preferably 120 mcg/kg/day. Please note, the said growth factors are for illustration only, and there are no meaning to limit the scope of growth factor for neural cell differentiation within these two growth factors. One skilled in the art could use other growth factor to induce mesenchymal cells from bone marrow. In one embodiment, the said neurological treatment with bone marrow transplantation is repeated three times every three months according to patient's recovery.

In another embodiment, for diabetes, the bone marrow solution is transfused with insulin-like growth factor-1 (IGF-1), and/or hepatocyte growth factor (HGF) instead of growth factors for neural differentiation, which promote mesenchymal stem cells in bone marrow to differentiate into insulin-secret cells, and replace the function in pancreas. The dose of IGF-1 is around 1 to 2 mg/kg. The daily dosage of HGF is in the range of 0.01 μg-10 mg per 1 kg of the body weight in an adult, preferably in the range of 0.1 μg-1 mg/kg, The said growth factors are just for illustration only, and there is no meaning to limit the scope of growth factor for differentiation by these said growth factors. One skilled in the art could use other growth factor to perform the same function. In addition, mannitol medication is not necessary.

Those skilled in the art will appreciate that numerous other embodiments and modifications are contemplated by the present invention. The above description of embodiments is merely illustrative and not intended to limit the scope of the present invention. The patents, literature, and references cited herein are incorporated by reference in their entireties.

### EXAMPLES

The following Examples are provided for illustrative purposes only and are not to be interpreted as limiting the scope of the present invention in any manner.

### Example 1 Stroke

A patient from Mexico had a stroke history for 10 years. Before bone marrow transplantation, she felt tired and dizzy frequently. Usually, her blood pressure went up to 240/120, which caused her dizzy. The patient could not lift her left arm up more than ½ inch. The left side of her body became partially paralyzed. The progression was slow and each day it got worse to this point where she could not stand up by herself.

The patient went to our affiliated hospital located in China, and she was pretreated with NEUPOGEN at the dose of 10 mcg/kg/day for continuous 4 days. Two days before bone marrow transplantation, mannitol was given at the dose of 2 g/kg body weight in a 24 hour time period. The patient is under a close health care to monitor the blood pressure and other medical index.

The patient was partially numbed on her left leg. A slight skin insect was performed by a lancet. The tibia bone was drilled to open with a needle, and the bone marrow was aspirated with syringe rinsed by heparin. Totally around 120 ml bone marrow were collected, which included all the component of bone marrow soft tissue as a whole. The said bone marrow was immediately delivered into a plastic bag containing 1000 ml saline water, 1 mg NGF per kg body weight, and 2 g mannitol/kg body weight. Following that, the bone marrow solution was dropped back into patient within 2 hours, where the said bone marrow did not process any cell isolation and cell expansion in vitro.

Nine days after bone marrow transplantation, the patient was able to stand up, and she started to walk a few steps on her own. The same treatment was performed once after three months. Patient walked inside our affiliated hospital at her own although her gait was slow, and she needed some support when she went to stairs. After the second treatment, her speech was clearer than before, which she can not do it. And she can lift 3 pounds package from ground. Further follow up is under process.

### Example 2 Spinal Cord Injury

A 38 year old America patient was suffering from spinal cord injury since 2002. He fell off a boat into shallow water, and a metal part of the boat hurt his neck. He was underwater for several minutes. His spinal cord at C3-C4 was fracture, and the connection was not complete. He had minimal movement in both arms and in his left leg, and no movement whatsoever in his hands or right leg. He could not stand. He also had a great deal of pain in his neck, lower back, and left foot. His blood pressure was very low, such that he needed a pacemaker to prevent passing out. His vision was restricted to vague shapes in black and white. His hearing was overwhelmed with background noise. He had become overweight from lack of mobility, which further complicated efforts to stand on his own strength.

The first time of patient went to our affiliated hospital in China was in September, 2006. Before bone marrow transplantation, the patient received NEUPOGEN intramuscular at the dose of 10 mcg/kg/day for continuous 4 days. In addition, mannitol was adminstered at the dose of 2 g/kg body weight for 2 days to increase the permeability of blood-brain-barrier. The patient is under a close health care to monitor the blood pressure and other medical index.

The patient was partially numbed at the bottom of spinal cord. A slight skin insect was performed at the numb site just at the bottom of the spinal cord and close to right sacral bone by a lancet. The sacral bone was drilled to open with a needle, and the bone marrow was aspirated with syringe rinsed by heparin. Totally around 160 ml bone marrow were collected, which included all the component of bone marrow soft tissue as a whole. The said bone marrow was immediately delivered into a plastic bag containing 1000 ml saline water, 1 mg NGF per kg body weight, and 2 g mannitol/kg body weight. Following that, the bone marrow solution was dropped back into patient within 2 hours, where the said bone marrow did not process any cell isolation and cell expansion in vitro.

Fifteen days after bone marrow transplantation, the patient was able to vaguely see some people walking on the hallway, and he could see light flash. On occasionally he can see nurse face clearly in rehab center. He also obtained some strength of his hand, which helps him to drive his wheelchair, and he can also flex his wrists back and forth, which he can not do it before. One month later, the patient was able to read at close distance and watch television.

The patient obtained addition bone marrow transplantation every three months. Right now the patient can read newspaper, and write email by himself. He is able to walk across a swimming pool in 4½ feet of water with no assistance, and to stand up in 3½ feet of water for several minutes. Further follow up is under process.

### Example 3 Multiple Sclerosis

In May, 2006, our China affiliated hospital treated a multiple sclerosis patient from Switzerland. The patient complained that he had constant pain in his legs, trouble controlling his bladder, had to walk with a cane or support because of balance problems, could not lift his legs off the bed and his speaking was not completely fluent.

The patient was treated continuously with NEUPOGEN intramuscular at the dose of 10 mcg/kg/day for 4 days. Besides that, the patient was also received mannitol treatment for 2 days at the dose of 2 g/kg body weight for 2 days to increase the permeability of blood-brain-barrier. The patient is under a close health care to monitor the blood pressure and other medical index.

The patient was partially numbed at the bottom of spinal cord. A slight skin insect was performed at the numb site just at the bottom of the spinal cord and close to right sacral bone by a lancet. The sacral bone was drilled to open with a needle, and the bone marrow was aspirated with syringe rinsed by heparin. Totally around 160 ml bone marrow were collected, which included all the component of bone marrow soft tissue as a whole. The said bone marrow was immediately delivered into a plastic bag containing 1000 ml saline water, 1 mg NGF per kg body weight, and 2 g mannitol/kg body weight. Following that, the bone marrow solution was dropped back into patient within 2 hours, where the said bone marrow did not process any cell isolation and cell expansion in vitro.

One month after treatment, the patient did not have the problem of all pain in his legs and they stopped shaking. He gained control over his bladder. He could lift his legs off the bed and he could speak more clearly. On occasionally he can take a walk with a better gait than before. The patient had another 2 bone marrow treatment, and his condition was improved constantly. He can speak much more clearly than before, and he looked like younger. We will follow up with him to see if he has further improvements.

### Example 4 Diabetes

In January 2007, a local Chinese patient visited our affiliated hospital complaining sever diabetes. He was diagnosed as type 1 diabetes. His blood sugar level was around 12 mmol/l before meal. He had blood circulation was poor, which resulted in ulcer on both of his legs.

The patient was treated continuously with NEUPOGEN intramuscular at the dose of 10 mcg/kg/day for 4 days. No mannitol treatment was applied for this patient. The patient is under a close health care to monitor the blood pressure and other medical index.

Since the patient had poor blood circulation on his lower limb, the bone marrow was isolated from his sacral bone. The patient was partially numbed at the bottom of spinal cord. A slight skin insect was performed at the numb site just at the bottom of the spinal cord and close to right sacral bone by a lancet. The sacral bone was drilled to open with a needle, and the bone marrow was aspirated with syringe rinsed by heparin. Totally around 160 ml bone marrow were collected, which included all the component of bone marrow soft tissue as a whole. The said bone marrow was immediately delivered into a plastic bag containing 1000 ml saline water, and 1 mg IGF-1 per kg body weight. No mannitol was given to this patient. Following that, the bone marrow solution was dropped back into patient within 2 hours, where the said bone marrow did not process any cell isolation and cell expansion in vitro.

The patient only had one time bone marrow transplantation. Three days after treatment, patient's blood sugar level dropped to 9 mmol/l before meal, which is very close to normal blood sugar range. He only took one third amount of insulin as he did before. One month later, x-ray showed good blood circulation on both of his legs. His leg ulcer started to be healed.

What is claimed is:

1. A method of treating stroke patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

2. The method according to claim 1, wherein the said stroke patient is pretreated with granulocyte colony-stimulating factor (G-CSF), or other similar medication to help stem cell release from bone marrow for 1-4 days;

3. The method according to claim 1, wherein the said stroke patient is pretreated with mannitol or other similar medication to open blood-brain-barrier for 1-3 days;

4. The method according to claim 2, wherein the said stroke patient is pretreated with mannitol or other similar medication to open blood-brain-barrier for 1-3 days;

5. The method according to claim 1, wherein the said bone marrow is obtained from the same patient or from different donor;

6. The method according to claim 1, wherein the said bone marrow has an amount at least above 1 ml;

7. The method according to claim 1, wherein the said growth factors consist of neural growth factor (NGF) or fibroblast growth factor (FGF) or growth factor;

8. The method according to any one of claims 1-7, wherein the said bone marrow solution further includes mannitol;

9. The method according to any one of claims 1-8, further comprises the step of having additional two said bone marrow transplantation every 3 months;

10. A method of treating spinal cord injury patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

11. The method according to claim 10, wherein the said spinal cord injury patient is pretreated with granulocyte colony-stimulating factor (G-CSF), or other similar medication to help stem cell release from bone marrow for 1-4 days;

12. The method according to claim 10, wherein the said spinal cord injury patient is pretreated with mannitol or other similar medication to open blood-brain-barrier for 1-3 days;

13. The method according to claim 11, wherein the said spinal cord injury patient is pretreated with mannitol or other similar medication to open blood-brain-barrier for 1-3 days;

14. The method according to claim 10, wherein the said bone marrow is obtained from the same patient or from different donor;

15. The method according to claim 10, wherein the said bone marrow has an amount at least above 1 ml;

16. The method according to claim 10, wherein the said growth factors consist of neural growth factor (NGF) or fibroblast growth factor (FGF) or growth factor;

17. The method according to any one of claims 10-16, wherein the said bone marrow solution further includes mannitol;

18. The method according to any one of claims 10-17, further comprises the step of having additional two said bone marrow transplantation every 3 months;

19. A method of treating Parkinson disease patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

20. The method according to claim 19, wherein the said Parkinson patient is pretreated with granulocyte colony-stimulating factor (G-CSF), or other medication to help stem cell release from bone marrow for 1-4 days;

21. The method according to claim 19, wherein the said Parkinson patient is pretreated with mannitol or other similar medication to open blood-brain-barrier for 1-3 days;

22. The method according to claim 20, wherein the said Parkinson patient is pretreated with mannitol or other similar medication to open blood-brain-barrier for 1-3 days;

23. The method according to claim 19, wherein the said bone marrow is obtained from the same patient or from different donor;

24. The method according to claim 19, wherein the said bone marrow has an amount at least above 1 ml;

25. The method according to claim 19, wherein the said growth factors consist of neural growth factor (NGF) or fibroblast growth factor (FGF) or growth factor;

26. The method according to any one of claims 19-25, wherein the said bone marrow solution further includes mannitol;

27. The method according to any one of claims 19-26, further comprises the step of having additional two said bone marrow transplantation every 3 months;

28. A method of treating multiple sclerosis patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

29. The method according to claim 28, wherein the said multiple sclerosis patient is pretreated with granulocyte colony-stimulating factor (G-CSF), or other medication to help stem cell release from bone marrow for 1-4 days;

30. The method according to claim 28, wherein the said multiple sclerosis patient is pretreated with mannitol or other similar medication to open blood-brain-barrier for 1-3 days;

31. The method according to claim 29, wherein the said multiple sclerosis patient is pretreated with mannitol or other similar medication to open blood-brain-barrier for 1-3 days;

32. The method according to claim 28, wherein the said bone marrow is obtained from the same patient or from different donor;

33. The method according to claim 28, wherein the said bone marrow has an amount at least above 1 ml;

34. The method according to claim 28, wherein the said growth factors consist of neural growth factor (NGF) or fibroblast growth factor (FGF) or growth factor;

35. The method according to any one of claims 28-35, wherein the said bone marrow solution further includes mannitol;

36. The method according to any one of claims 28-36, further comprises the step of having additional two said bone marrow transplantation every 3 months;

37. A method of treating Amyotrophic Lateral Sclerosis patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

38. The method according to claim 37, wherein the said Amyotrophic Lateral Sclerosis patient is pretreated with granulocyte colony-stimulating factor (G-CSF), or other similar medication to help stem cell release from bone marrow for 1-4 days;

39. The method according to claim 37, wherein the said Amyotrophic Lateral Sclerosis patient is pretreated with mannitol or other similar medication to open blood-brain-barrier for 1-3 days;

40. The method according to claim 38, wherein the said Amyotrophic Lateral Sclerosis patient is pretreated with mannitol or other similar medication to open blood-brain-barrier for 1-3 days;

41. The method according to claim 37, wherein the said bone marrow is obtained from the same patient or from different donor;

42. The method according to claim 37, wherein the said bone marrow has an amount at least above 1 ml;

43. The method according to claim 37, wherein the said growth factors consist of neural growth factor (NGF) or fibroblast growth factor (FGF) or growth factor;

44. The method according to any one of claims 37-44, wherein the said bone marrow solution further includes mannitol;

45. The method according to any one of claims 37-45, further comprises the step of having additional two said bone marrow transplantation every 3 months;

46. A method of treating diabetes patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

47. The method according to claim 46, wherein the said diabetes patient is pretreated with granulocyte colony-stimulating factor (G-CSF), or other similar medication to help stem cell release from bone marrow for 1-4 days;

48. The method according to claim 46, wherein the said bone marrow is obtained from the same patient or from different donor;

49. The method according to claim 46, wherein the said bone marrow has an amount at least above 1 ml;

50. The method according to claim 46, wherein the said growth factors consist of insulin-like growth factor-1 (IGF-1), or hepatocyte growth factor (HGF) or growth factor;

51. The method according to any one of claims 46-50, further comprises the step of having additional two said bone marrow transplantation every 3 months;

52. A method of preventing brain injury with bone marrow comprises steps of:
1) having a patient with a head concussion within 1 day to 1 month;
2) obtaining bone marrow;
3) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
4) transfusing the said bone marrow solution into stroke patient;

53. The method according to claim 52, wherein the said patient is pretreated with granulocyte colony-stimulating factor (G-CSF), or other similar medication to help stem cell release from bone marrow for 1-4 days;

54. The method according to claim 52, wherein the said patient is pretreated with mannitol or other similar medication to open blood-brain-barrier for 1-3 days;

55. The method according to claim 53, wherein the said patient is pretreated with mannitol or other similar medication to open blood-brain-barrier for 1-3 days;

56. The method according to claim 52, wherein the said bone marrow is obtained from the same patient or from different donor;

57. The method according to claim 52, wherein the said bone marrow has an amount at least above 1 ml;

58. The method according to claim 52, wherein the said growth factors consist of neural growth factor (NGF) or fibroblast growth factor (FGF) or growth factor;

59. The method according to any one of claims 52-58, wherein the said bone marrow solution further includes mannitol;

60. The method according to any one of claims 52-59, further comprises the step of having additional two said bone marrow transplantation every 3 months;

61. A method of preventing spinal cord injury with bone marrow comprises steps of:
1) having a patient with a vertebrate hurt within 1 day to 1 month;
2) obtaining bone marrow;
3) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
4) transfusing the said bone marrow solution into stroke patient;

62. The method according to claim 61, wherein the said patient is pretreated with granulocyte colony-stimulating factor (G-CSF), or other similar medication to help stem cell release from bone marrow for 1-4 days;

63. The method according to claim 61, wherein the said patient is pretreated with mannitol or other similar medication to open blood-brain-barrier for 1-3 days;

64. The method according to claim 62, wherein the said patient is pretreated with mannitol or other similar medication to open blood-brain-barrier for 1-3 days;

65. The method according to claim 61, wherein the said bone marrow is obtained from the same patient or from different donor;

66. The method according to claim 61, wherein the said bone marrow has an amount at least above 1 ml;

67. The method according to claim 61, wherein the said growth factors consist of neural growth factor (NGF) or fibroblast growth factor (FGF) or growth factor;

68. The method according to any one of claims 61-67, wherein the said bone marrow solution further includes mannitol;

69. The method according to any one of claims 61-68, further comprises the step of having additional two said bone marrow transplantation every 3 months;

70. A method of treating Cerebral palsy patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

71. The method according to claim 70, wherein the said Cerebral palsy patient is pretreated with granulocyte colony-stimulating factor (G-CSF), or other similar medication to help stem cell release from bone marrow for 1-4 days;

72. The method according to claim 70, wherein the said Cerebral palsy patient is pretreated with mannitol or other similar medication to open blood-brain-barrier for 1-3 days;

73. The method according to claim 71, wherein the said Cerebral palsy patient is pretreated with mannitol or other similar medication to open blood-brain-barrier for 1-3 days;

74. The method according to claim 70, wherein the said bone marrow is obtained from the same patient or from different donor;

75. The method according to claim 70, wherein the said bone marrow has an amount at least above 1 ml;

76. The method according to claim 70, wherein the said growth factors consist of neural growth factor (NGF) or fibroblast growth factor (FGF) or growth factor;

77. The method according to any one of claims 70-77, wherein the said bone marrow solution further includes mannitol;

78. The method according to any one of claims 70-78, further comprises the step of having additional two said bone marrow transplantation every 3 months;

79. A method of treating Alzheimer's disease patient with bone marrow comprises steps of:
1) obtaining bone marrow;
2) delivering the said bone marrow as a whole into a plastic bag containing saline water (or glucose solution or other medical solution) and growth factors, which become a bone marrow solution. The said bone marrow does not need further cell isolation and cell expansion, and it is used as a whole in the process;
3) transfusing the said bone marrow solution into stroke patient;

80. The method according to claim 79, wherein the said Alzheimer's disease patient is pretreated with granulocyte colony-stimulating factor (G-CSF), or other similar medication to help stem cell release from bone marrow for 1-4 days;

81. The method according to claim 79, wherein the said Alzheimer's disease patient is pretreated with mannitol or other similar medication to open blood-brain-barrier for 1-3 days;

82. The method according to claim 80, wherein the said Alzheimer's disease patient is pretreated with mannitol or other similar medication to open blood-brain-barrier for 1-3 days;

83. The method according to claim 79, wherein the said bone marrow is obtained from the same patient or from different donor;

84. The method according to claim 79, wherein the said bone marrow has an amount at least above 1 ml;

85. The method according to claim 79, wherein the said growth factors consist of neural growth factor (NGF) or fibroblast growth factor (FGF) or growth factor;

86. The method according to any one of claims 79-86, wherein the said bone marrow solution further includes mannitol;

87. The method according to any one of claims 70-87, further comprises the step of having additional two said bone marrow transplantation every 3 months;
